(19) United States
(12) Patent Application Publication
(10) Pub. No.: US 2021/0257079 A1
(43) Pub. Date: Aug. 19, 2021

(54) RECOMMENDATION DEVICES AND METHODS FOR NUTRITION-RELATED COMMODITY

(71) Applicant: Yukashikado Inc., Tokyo (JP)

(72) Inventors: Shinya MINOBE, Tokyo (JP); Keiichiro SAITO, Tokyo (JP)

(21) Appl. No.: 17/144,279

(22) Filed: Jan. 8, 2021

(30) Foreign Application Priority Data
Jan. 8, 2020 (JP) 2020-1748
Aug. 5, 2020 (JP) 2020-133278

(51) Int. Cl. G16H 20/60; G16H 10/20; G06Q 30/06; G01N 33/493

(52) U.S. Cl. CPC G16H 20/60; G16H 10/20; G01N 33/493; G06Q 30/0633; G06Q 30/0631

(57) ABSTRACT

A nutrition-related commodity recommendation device is provided that proposes a product that matches nutritional state of users and promotes user's willingness to purchase. The device comprising: biological information acquiring means for acquiring biological information of users; question information acquiring means for acquiring question information of users; nutritional information calculation means for calculating nutritional information related to excess and deficiency of nutrients in bodies of users based on biological information and question information; related commodity extraction means for extracting a commodity group related to excess and deficiency nutrients based on the calculated nutritional information; inappropriate commodity exclusion means for excluding inappropriate commodities that are not suitable for users from the commodity group; priority setting means for setting a priority for each commodity of the commodity group from which inappropriate commodities are excluded; commodity determining means for determining proposed commodities based on the priority; and proposed commodity output means for outputting data related to proposed commodities.

### TECHNICAL FIELD

The present invention relates to a technique for proposing a nutrition-related commodity based on biological information and the like.

### BACKGROUND ART

In recent years, the number of people who consume health foods and supplements has increased due to increased health consciousness. However, since it is difficult for general users to accurately grasp the nutrients lacking in the users, and many of the users do not have specialized knowledge on nutrition, it is actually the situation that the user selects and ingests supplements or the like based on his/her own experience or the like (see Patent Document 1).

Therefore, it is known a supplement purchasing system in which a user's urine is analyzed using a terminal of an analysis organization, vitamins, minerals, and the like lacking in the user are pointed out from the analysis result, and a supplement name for recommending purchasing is transmitted to the terminal of the user.

In the supplement purchasing system disclosed in Patent Document 1, not only a desired supplement can be selected from among recommended supplements and transmitted to a terminal of a manufacturing organization, but also a situation in which a urine collection container is sent can be transmitted to a terminal of a user, so that the system has a function of prompting periodic inspection.

With such a system, the user can accurately grasp the nutrients lacking in the user, and purchase and ingest a supplement suitable for the user.

However, although supplements supplement the nutritional deficiency of the user, they are not themselves delicious, and food commodities such as health foods do not necessarily taste in accordance with the taste of the user. Therefore, even if a supplement or the like suitable for the user is proposed, the user cannot purchase the supplement unless the user's willingness to purchase the supplement or the like increases, resulting in a problem that the user's health cannot be promoted.

As a technique for proposing a supplement suitable for a user, a supplement proposal system is known which includes a supplement information storage unit that stores a recommendation level of a supplement for a user's trouble, and narrows down the proposed supplement based on the trouble received from the user and the degree of the trouble (see patent document 2).

According to the supplement proposal system, it is possible to propose supplements according to the user's worries such as diet, anti-aging of skin, and recovery from fatigue, and it is possible to enhance the user's willingness to purchase the supplement.

However, in order to maximize the user's willingness to purchase, it is not sufficient to solve the user's trouble. In other words, in order for the user to lead a healthy and rich life both physically and mentally, it is not necessary to supplement the deficient nutrients or solve the user's troubles, but it is necessary to propose commodities that the user can actively take interest in, taste, and enjoy. However, the supplement proposal system described above has a problem in that it is not possible to propose commodities that can be actively interested by the user regardless of the trouble or the like.

Further, since the supplement proposing system disclosed in patent document 2 identifies an insufficient nutrient based on the inquiry information and does not utilize biological information such as urine, there is a problem that an accurate identification of an insufficient nutrient is not made.

### PRIOR ART

### Patent Documents

[Patent Document 1] JP 2007-87192 A

[Patent Document 2] JP 6586499 B

### OUTLINE OF THE INVENTION

### Problems to be Solved by the Invention

In view of such circumstances, it is an object of the present invention to provide a nutrition-related commodity recommendation device and method for proposing a commodity that matches the nutrition state of a user and promotes the user's willingness to purchase.

In order to solve the above-mentioned problems, a nutrition-related commodity recommendation device of the present invention is the device for proposing commodities related to nutritional conditions of users, and includes the following means 1) to 8).

1) A biological information acquiring means for acquiring biological information of the user.

2) A question information acquiring means for acquiring the user's question information.

3) A nutrition information calculation means for calculating nutritional information on excess and deficiency of nutrients in the user's body based on biological information and question information.

4) A related commodity extraction means for extracting commodity groups related to excess and deficiency nutrients based on the calculated nutritional information.

5) An inappropriate commodity exclusion means for excluding inappropriate commodities that are not suitable for the user from the commodity groups.

6) A priority setting means for setting a priority for each commodity in the commodity group from which inappropriate the commodities have been excluded.

7) A commodity determining means for determining proposed commodities on a basis of the priority.

8) A proposed commodity output means for outputting data relating to the proposed commodities.

1) The biological information in the biological information acquiring means broadly includes biological information that can be collected from a human body such as urine, saliva, sweat, blood, stool, nails, hair, or skin, but is preferably body fluid component information of a user obtained by an examination including a urine examination, and more preferably is body fluid component information of a user obtained by a urine examination. As a method of acquiring urine component information, for example, urine collected using a test kit may be sent by mail to measure the component, or the component may be measured by a simple test using a test paper.

2) The question information in the question information acquiring means is the content of the user's answer to the question, and includes personal information of the user such as name, address, date of birth, etc., and information such as smoking, drinking, allergic constitution, presence or absence of chronic disease, etc.

3) In the nutritional information calculation means, it is preferable that the nutritional information is information relating to nutrients including vitamins and minerals, but is not limited to the above nutrients. The vitamins here are specifically fat-soluble vitamins such as vitamin A, vitamin D, vitamin E, and vitamin K, and water-soluble vitamins such as group B vitamins and vitamin C, and the six types of group B vitamins are vitamin B1, vitamin B2, vitamin B6, niacin, pantothenic acid, and biotin. Group B vitamins are essential nutrients for the production of animal energy and are known to be involved in the metabolism of carbohydrates, fats, and proteins, in cooperation with each other, and in the metabolism of various substances. Therefore, by examining the urine concentration of group B vitamins, it is possible to clarify the deficient nutrients in the user's body.

Also, minerals are sodium, magnesium, potassium, calcium, phosphorus, sulfur, chlorine, cobalt, zinc, iron, copper, selenium, manganese, chromium, molybdenum, iodine, and the like in urine.

Examples of nutrients other than vitamins and minerals include essential fatty acids such as ω-6 fatty acids and ω-3 fatty acids.

4) In the related commodity extraction means, commodities are narrowed down based on excess and deficiency nutrients from all commodities that can be proposed by the service provider. Specifically, for example, a food containing a nutrient supplementing a deficient nutrient is extracted. It is also possible to extract foods which not only supplement deficient nutrients but also have the effect of excreting excessively ingested nutrients or foods which do not contain excessively ingested nutrients.

Note that, in this specification, a commodity is not limited to an article such as a food or a supplement, and is used in the sense of including a service. Therefore, for example, it is possible to propose coupons that can be used at convenience stores, dispensing pharmacies, drug stores, restaurants, and exercise facilities.

5) The inappropriate commodity exclusion means preferably excludes the commodity based on the past proposal history and the commodity purchase history of the user, and more preferably excludes the commodity based on at least one of the allergy content of the user, the age of the user, and the gender of the user. Further, the present invention is not limited to the above-mentioned items, and for example, when the user selects “no supplement” when the question information is provided, it is possible to exclude the supplement.

The exclusion based on the past proposal history and the merchandise purchase history is to exclude the merchandise proposed to the user or the merchandise purchased by the user within a predetermined period of time. Exclusion based on the user's age is to exclude, for example, generation-specific items if they do not match the user's generation, and exclusion based on the user's gender is to exclude items if, for example, gender-specific items do not match the user's gender. In addition, 3) commodities containing nutrients judged to be excessive in the nutrition information calculation means can also be excluded.

6) The priority setting means uses an element considered to attract the user's interest as evaluation items and sets the priority for each commodity by using weightings thereof, and preferably sets the priority for each commodity by using the evaluation items of at least one of the temporal food classification, new commodities, favorite commodities, sellable commodities, recommended commodities, strategic commodities, seasonal-limited commodities, and sales commodities, and weightings thereof, and further preferably sets the priority for each commodity by using the evaluation item of at least one of the search target commodities by the user, the commodities having the same attributes as the commodities purchased by the user, and the purchase cancellation commodities by the user, and weightings thereof. In addition, review evaluation, that is, for example, five-level evaluation from a user which is not limited to the user may be used as evaluation items.

7) Preferably, the commodity determining means determines the number of items of the proposed commodity and the order of the proposals. The number of items of commodities is the number of types of commodities proposed to the user, and the order of proposal of commodities is, for example, the order of commodities displayed on the display of the terminal held by the user, and the priority order of proposals can be determined in such a manner that commodities to be displayed with higher priority are displayed. In determining the proposed priority order, 6) it can be used in combination with the priority setting result made in the priority setting means. As an example of the order of proposal of commodities, there is an order of proposal based on a combination of dishes or the like, such as the order of main dishes, main foods, side dishes, seasonings, and juice. Here, the main dish, the staple dish, the side dish, or the juice may include a food used for the main dish, the staple dish, the side dish, or the juice, such as a seasoning or a supplement, or the seasoning or the like may be handled as an attribute different from the main dish or the like as described above.

Therefore, for example, when 6) the number of items of commodities for which priority is set in the priority setting means is 10 and the number of items of commodities to be proposed is 4, 7) the commodity determining means can select and determine four commodities from the top according to the order of proposal of the commodities and the priority setting.

8) As the proposed commodity output means, it is preferable to display the proposal result on the display of the terminal held by the user via the communication means such as the Internet, but for example, the proposal result may be displayed on the display of the terminal held by the service provider, or the proposal result may be printed out and provided to the user by mail or the like. In addition, in a cash register installed in a store such as a convenience store, the proposal result may be displayed on a terminal that can be operated or confirmed by the user, such as displayed on a display displayed on the customer side.

The nutrition-related commodity recommendation method of the present invention is a method of proposing a commodity related to a nutritional state of a user, comprising the following 1) to 8) steps:

1) A biological information acquiring step for acquiring biological information of a user.

2) A question information acquiring step for obtaining the user's question information.

3) A nutrition information calculation step for calculating nutritional information on excess and deficiency of nutrients in a user's body based on biological information and question information.

4) A related commodity extraction step for extracting a commodity group related to an excess or deficiency nutrient based on the calculated nutritional information.

5) An inappropriate commodity exclusion step for excluding inappropriate commodities that are not suitable for the user from the commodity group.

6) A priority setting step for setting the priority for each commodity in the group of commodities from which inappropriate commodities have been excluded.

7) A commodity determining step for determining proposed commodities based on priority.

8) A proposal commodity output step for outputting data on proposed commodities.

The nutrition-related commodity recommendation program of the present invention is for causing a computer to perform 3) the nutrition information calculation step, 4) the related commodity extraction step, 5) the inappropriate commodity exclusion step, 6) the priority setting step, and 7) the commodity determining step in the above-described nutrition-related commodity recommendation method.

In the nutrition-related commodity recommendation system of the present invention, in a system in which a client terminal on the user side and a server on the service provider side are connected via a network to transmit and receive data, the server includes any of the nutrition-related commodity recommendation devices described above, and the client terminal includes means for inputting question information of the user and means for displaying a proposal commodity on a display.

With the above-described configuration, the user can easily receive a proposal of a commodity suitable for himself/herself by using a hand-held client terminal. As the client terminal, a wide range of terminals such as a smart phone, a tablet terminal, and a PC can be used.

In the nutrition-related commodity recommendation system of the present invention, it is preferable that the server further comprises means for automatically adding the proposed commodity to the cart of the user in the electronic commerce, and the client terminal further comprises commodity changing means for excluding unnecessary commodities from the proposed commodities automatically added to the cart, or for manually adding necessary commodities to the cart. By providing means for automatically adding the proposed commodity to the cart, the user's labor can be saved and the convenience can be improved. Also, by providing a commodity changing means, the user can easily select a commodity suitable for the user. The automatic addition to the cart is preferably performed within a predetermined amount, for example, “the total amount is 3800 to 4200 yen”.

In the nutrition-related commodity recommendation system of the present invention, it is preferable that the client terminal further comprises regular delivery service application means for applying for periodic delivery of the commodity, and the server further comprises regular delivery service approval means for accepting application for regular delivery service by the user, and means for delivering the commodity on a predetermined date or every elapse of a predetermined period.

Since the client terminal includes the means for applying for the regular delivery service and the server includes the means for accepting the regular delivery service, the user can use the regular delivery service through a simple procedure. Here, the term “regular” means, for example, every month, every week, or the like. When commodities are sent on a regular daily basis, for example, they are sent on the 1st day of each month. When goods are sent on a regular basis, they are sent every week. In the regular delivery service application means, it is preferable that the user can freely set the frequency of sending the commodities.

### Effects of the Invention

According to the nutrition-related commodity recommendation device and method of the present invention, it is possible to propose a commodity which is adapted to a nutritional state of a user and which promotes a user's willingness to buy.

### BRIEF DESCRIPTION OF THE DRAWINGS

FIG. 1 shows a functional block diagram of a nutrition-related commodity recommendation device.

FIG. 2 shows a schematic flow chart of a nutrition-related commodity recommendation method.

FIG. 3 shows a priority setting flow chart of a nutrition-related commodity recommendation method.

FIG. 4 shows a commodity determining flow chart of a nutrition-related commodity recommendation method.

FIG. 5 shows a screen display image of a user terminal.

FIG. 6 shows a schematic block diagram of a nutrition-related commodity recommendation system of Embodiment 1.

FIG. 7 shows a functional block diagram of a nutrition-related commodity recommendation system of Embodiment 1.

FIG. 8 shows a functional block diagram of a nutrition-related commodity recommendation system of Embodiment 2.

FIG. 9 shows a Schematic flow diagram of a nutrition-related commodity recommendation system of Embodiment 2.

### MODE FOR CARRYING OUT THE INVENTION

Embodiments of the present invention will be described in detail below with reference to the drawings. The present invention is not limited to the following embodiments and examples shown in the figures, and the present invention can be variously changed in design.

### Embodiment 1

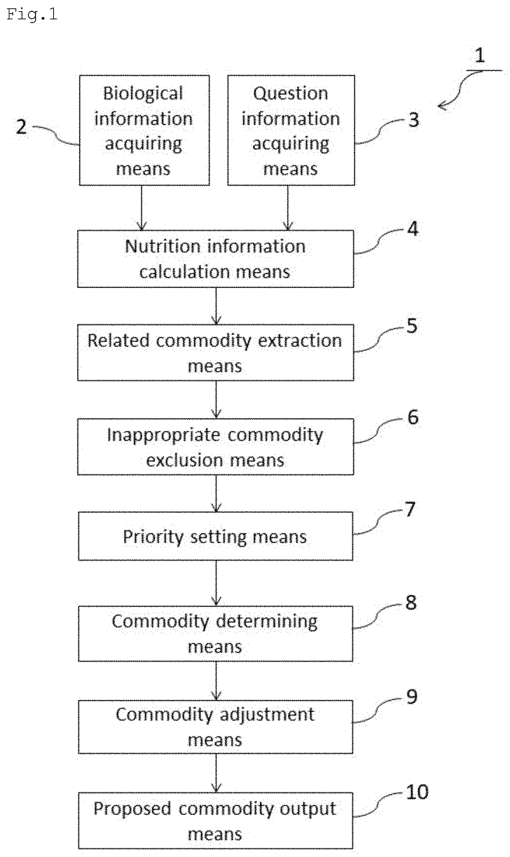

FIG. 1 shows a functional block diagram of the nutrition-related commodity recommendation device. As shown in FIG. 1, the nutrition-related commodity recommendation device 1 includes a biological information acquiring means 2, a question information acquiring means 3, a nutrition information calculation means 4, a related commodity extraction means 5, an inappropriate commodity exclusion means 6, a priority setting means 7, a commodity determining means 8, a commodity adjustment means 9, and a proposed commodity output means 10.

The biological information acquiring means 2 acquires biological information of the user, and in the present embodiment, acquires urine component information of the user obtained by the urine test. As a method of acquiring urine component information, a method of measuring a component by sending urine collected by mail using a test kit is used. The question information acquiring means acquires information on the user in a form of answering a questionnaire, and may respond with “Yes” or “No”, for example, or may enter or input specific contents. The nutritional information calculating means 4 calculates nutrients such as vitamins and minerals lacking in the body of the user based on the biological information acquired from the biological information acquiring means 2 and the question information acquired from the question information acquiring means 3.

The related commodity extraction means 5 narrows down the commodities based on the excess and deficiency nutrients from all the commodities that can be proposed by the service provider (not shown) using the nutrition-related commodity recommendation device 1. The inappropriate commodity exclusion means 6 excludes commodities which are determined to be inappropriate for the user. The priority setting means 7 performs weightings of commodities by using an element that is considered to attract the user's interest as evaluation items. The commodity determining means 8 determines the number of items of the proposed commodity and the order of proposals, and the commodity adjusting means 9 adds, changes, and deletes items of the proposed commodity and changes the order of proposals and designs by the hand of the service provider.

The proposed commodity output means 10 outputs data relating to the proposed commodity, and in this embodiment, the suggested result is displayed on the display of the terminal held by the user via the Internet.

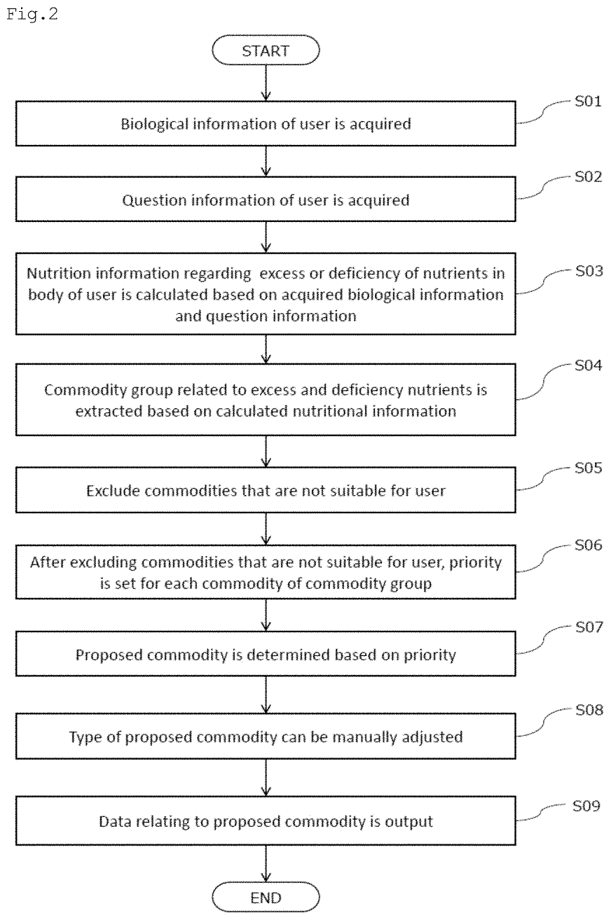

Here, a flow of a nutrition-related commodity recommendation method will be described. FIG. 2 shows a schematic flow diagram of a nutrition-related commodity recommendation method. As shown in FIG. 2, first, the biological information of the user is acquired by the biological information acquiring means 2 (step S01: Biological information acquiring step). Next, the user's question information is acquired (step S02: Question information acquiring step). Although the question information is acquired (step S02) after the biological information of the user is acquired (step S01), the biological information may be acquired (step S01) after the question information is acquired (step S02).

Nutrition information regarding the excess or deficiency of nutrients in the body of the user is calculated based on the acquired biological information and question information (step S03: Nutrition information calculation step). A commodity group related to the excess and deficiency nutrients is extracted based on the calculated nutritional information (step S04: Related commodity extraction step). Exclude commodities that are not suitable for the user from the commodity group (step S05: Inappropriate commodity exclusion step).

(Priority Setting Step)

After excluding commodities that are not suitable for the user, the priority is set for each commodity of the commodity group (step S06: Priority setting step). Here, a method of setting the priority will be described. First, it is assumed that, based on the calculated nutritional information, a group of commodities related to the excess and deficiency nutrients is extracted (step S04), and commodities unsuitable for the user are excluded (step S05), and as a result, 10 types of commodities are narrowed down to foods (A to D), health foods (E to G), and supplements (H to J).

Food (A to D) refers to staple foods such as rice and bread, foods such as meat, fish, eggs, and soybeans that are cooked as main vegetables, and foods such as vegetables that are cooked as side vegetables. Specifically, foods A and B are foods that are main foods, food C is foods that are main vegetables, and food D is foods that are side vegetables.

Health foods (E to G) are foods containing, for example, chlorella, aloe, protein, enzyme, collagen, ginseng, royal jelly, and the like, and include foods for specified health use, foods with nutritional functions, and foods with functional labeling. Specifically, health foods E are foods that can be used as staple foods, health foods F are foods that can be used as main vegetables, and health foods G are foods that can be used as side vegetables.

Supplements are concentrated, tablet, capsule, or powder of vitamins, minerals, and other ingredients.

The priority setting means 7 of the present embodiment has a total of 11 types of evaluation items from No. 1 to 11, and each item satisfying these evaluation items is assigned one point, and as a result of calculating the total points of each item, the item that has obtained the highest point is set as the item having the highest priority.

The temporal food classification of the evaluation item No. 1 is established so as not to be prioritized for commodities suitable for breakfast, lunch, snack, or dinner. In the first determination, the temporal food classification of the item No. 1 is determined to be “suitable for breakfast” in the second determination, “suitable for lunch” in the third determination, is determined to be “suitable for snack” and in the fourth determination, it is determined to be “suitable for dinner”.

It should be noted that the commodities set to have the highest priority in the first determination are excluded from the object of determination in the second determination, the commodities set to have the highest priority in the first and second determinations are excluded from the object of determination in the third determination, and the commodities set to have the highest priority in the first to third determinations are excluded from the object of determination in the fourth determination.

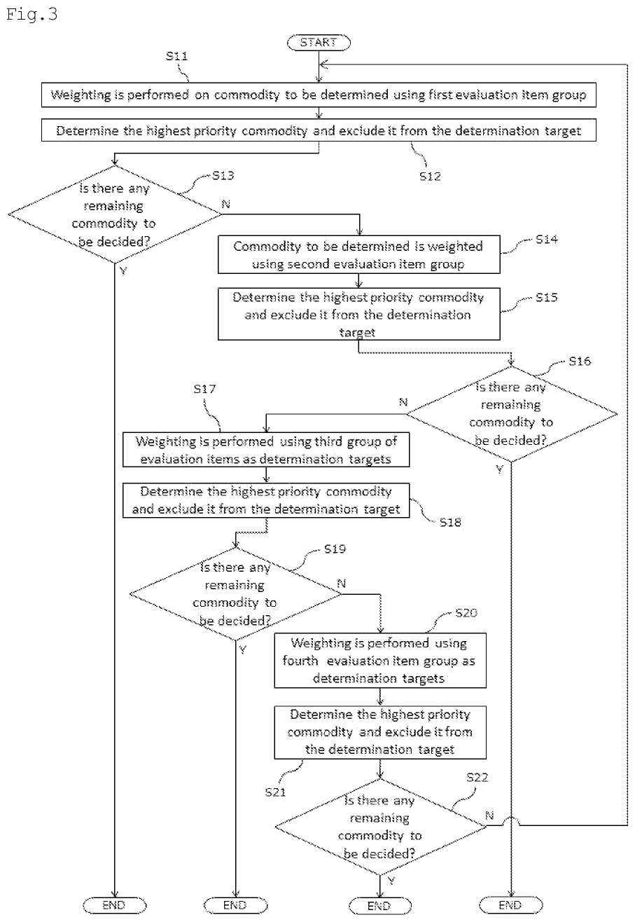

FIG. 3 shows a priority setting flow diagram of a nutrition-related commodity recommendation method. As shown in FIG. 3, first, weighting is performed on the commodity to be determined using the first evaluation item group (step S11). The commodity with the highest priority is determined and excluded from the determination targets (step S12). Table 1 below shows a determination image when the first determination is performed using the first evaluation item group.

TABLE 1

| | | | | | | Health | Health | Health | Supplement | Supplement | Supplement |
|---|---|---|---|---|---|---|---|---|---|---|---|
| No. | Evaluation items | Food A | Food B | Food C | Food D | food E | food F | food G | H | I | J |
| 1 | Temporal food | 1 | 0 | 0 | 1 | 0 | 1 | 0 | 0 | 0 | 0 |
| | classification (Breakfast) | | | | | | | | | | |
| 2 | New commodities | 1 | 0 | 0 | 0 | 1 | 0 | 0 | 0 | 0 | 0 |
| 3 | Favorite commodities | 1 | 0 | 0 | 0 | 0 | 1 | 0 | 0 | 1 | 0 |
| 4 | Good-selling | 0 | 1 | 0 | 0 | 1 | 1 | 0 | 0 | 1 | 1 |
| | commodities | | | | | | | | | | |
| 5 | Recommended | 1 | 0 | 0 | 0 | 0 | 0 | 1 | 0 | 1 | 1 |
| | commodities | | | | | | | | | | |
| 6 | Search history | 0 | 0 | 1 | 0 | 0 | 1 | 0 | 0 | 1 | 0 |
| 7 | Review evaluation | 1 | 1 | 0 | 0 | 0 | 1 | 0 | 1 | 0 | 0 |
| 8 | Seasonal-limited | 0 | 1 | 1 | 0 | 0 | 0 | 0 | 0 | 0 | 0 |
| | commodities | | | | | | | | | | |
| 9 | Identical attribute | 0 | 0 | 0 | 1 | 0 | 1 | 0 | 0 | 1 | 0 |
| | purchased commodities | | | | | | | | | | |
| 10 | Purchase canceled | 0 | 0 | 1 | 0 | 0 | 0 | 1 | 1 | 0 | 0 |
| | commodities | | | | | | | | | | |
| 11 | Strategic commodities | 0 | 1 | 0 | 0 | 0 | 1 | 1 | 0 | 0 | 0 |

| | | | | | | | | | | |
|---|---|---|---|---|---|---|---|---|---|---|
| Total | 5 | 4 | 3 | 2 | 2 | 7 | 3 | 2 | 5 | 2 |

As shown in Table 1 above, the evaluation items include a temporal food classification (No. 1), new commodities (No. 2), favorite commodities (No. 3), good-selling commodities (No. 4), recommended commodities (No. 5), search history (No. 6), review evaluation (No. 7), seasonal-limited commodities (No. 8), identical attribute purchased commodities (No. 9), purchase canceled commodities (No. 10), and strategic commodities (No. 11).

In the first evaluation item group, the temporal food classification (No. 1) is regarded as “suitable for breakfast”. New commodities (No. 2) refer to the time when the relevant commodity is released within the predetermined period.

The favorite commodities (No. 3) are commodities in which the user has already registered as favorite commodities, and the good-selling commodities (No. 4) are commodities in which all users or a certain range of users sold well within a predetermined period of time, not limited to the user. If the commodity proposed to the user has already been registered as the favorite commodities No. 3, it is highly likely that the user is interested in the commodity. Even if the commodity is not a favorite commodity, if the commodity is a new commodity (No. 2) or good-selling commodity (No. 4), it can be said that it attracts the eyes of the user and raises the willingness to purchase the commodity.

The recommended commodities No. 5 are, for example, commodities or the like for which recommendation is determined after the service provider confirms taste, quality, and the like. When the staff tastes seasonal fruits and the like and intends to propose them because they are very delicious, the user can attract the user's interest as a commodity recommended by the staff by making it a recommended commodity (No. 5).

The search history No. 6 is used to determine whether or not the commodity has been searched for by the user in the past. This is because it is considered that the user is likely to have an interest in the commodity when the history of the user's search in the past exists.

The review evaluation No. 7 is, for example, not limited to the user, but is a commodity that has been evaluated by the user in five grades or more. This is because commodities with good review evaluation (No. 7) are likely to be good-selling commodities.

Seasonal-limited commodities (No. 8) are commodities sold only for a certain period of the season. Seasonally perceived merchandise attracts the user's interest, and limited sales periods can enhance buying motivation.

The identical attribute purchased commodities No. 9 satisfy the evaluation item when the user and the user have the same attribute such as gender or generation, for example. For example, there are commodities that are good-selling for women and commodities that are good-selling for the elderly, and there is a high possibility that the user is also interested in the commodities that are often purchased with the same attributes as the user.

The purchase cancelled commodities (No. 10) are cart abandonments, and are commodities which are once considered to be purchased but are subsequently cancelled to be purchased. Although the merchandise deleted from the cart has not been purchased, it is highly likely that the user is interested in the merchandise.

Strategic commodities (No. 11) are commodities purchased in large quantities by service providers and commodities purchased at low cost. Since the service provider may purchase a large amount of recommended commodities (No. 5), such commodities are also used as the evaluation items.

As shown in Table 1 above, for Food A, a total of 5 points are assigned to the temporal food classification (No. 1), the new commodities (No. 2), the favorite commodities (No. 3), the recommended commodities (No. 5), and the review evaluation (No. 7) in order to satisfy the evaluation items. Food B is given a total of four points to satisfy the evaluation items for good-selling commodities (No. 4), review evaluation (No. 7), seasonal-limited commodities (No. 8) and strategic commodities (No. 11). Food C is given a total of three points to satisfy the evaluation items for search history (No. 6), seasonal-limited commodities (No. 8) and purchase cancelled commodities (No. 10). Food D is given a total of two points to satisfy the evaluation items for temporal food classification (No. 1) and identical attribute purchased commodities (No. 9).

For health foods E, a total of 2 points are assigned to satisfy the criteria for new commodities (No. 2) and good-selling commodities (No. 4). For health foods F, a total of 7 points are assigned to satisfy the criteria for temporal food classification (No. 1), favorite commodities (No. 3), good-selling commodities (No. 4), search history (No. 6), review evaluation (No. 7), identical attribute purchased commodities (No. 9), and strategic commodities (No. 11). For health foods G, a total of 3 points are assigned to satisfy the criteria for recommended commodities (No. 5), cancelled commodities (No. 10), and strategic commodities (No. 11).

For supplement H, a total of 2 points are assigned to satisfy the criteria for review evaluation (No. 7) and purchase canceled commodities (No. 10). For supplement I, a total of 5 points are assigned to satisfy the criteria for favorite commodities (No. 3), good-selling commodities (No. 4), recommended commodities (No. 5), search history (No. 6), and identical attribute purchased commodities (No. 9). For supplement J, a total of 2 points are assigned to satisfy the criteria for good-selling commodities (No. 4) and recommended commodities (No. 5).

As described above, the health food F having the highest total point of 7 points is regarded as the commodity having the highest priority and is excluded from the determination target (step S12).

It should be noted that, if a commodity that is not suitable for the user is excluded in step S05 and is narrowed down to only one type of commodity, the commodity is excluded in step S12, so that the commodity to be determined does not remain, and the priority setting is completed (step S13). On the other hand, when the commodity to be determined remains, the commodity to be determined is weighted using the second evaluation item group (step S14).

In the present embodiment, since nine types of commodities, i.e., foods (A to D), health foods (E, G), and supplements (H to J), remain, the commodities are weighted using the second group of evaluation items as determination targets (step S14). In the same manner as in the first determination, the commodity having the highest priority is determined and excluded from the determination targets (step S15). Table 2 below shows a determination image when the second determination is performed using the second evaluation item group.

TABLE 2

| | | | | | | Health | Health | Supplement | Supplement | Supplement |
|---|---|---|---|---|---|---|---|---|---|---|
| No. | Evaluation items | Food A | Food B | Food C | Food D | food E | food G | H | I | J |
| 1 | Temporal food | 0 | 0 | 0 | 0 | 1 | 0 | 0 | 0 | 0 |
| | classification (Lunch) | | | | | | | | | |
| 2 | New commodities | 1 | 0 | 0 | 0 | 1 | 0 | 0 | 0 | 0 |
| 3 | Favorite commodities | 1 | 0 | 0 | 0 | 0 | 0 | 0 | 1 | 0 |
| 4 | Good-selling | 0 | 1 | 0 | 0 | 1 | 0 | 0 | 1 | 1 |
| | commodities | | | | | | | | | |
| 5 | Recommended | 1 | 0 | 0 | 0 | 0 | 1 | 0 | 1 | 1 |
| | commodities | | | | | | | | | |
| 6 | Search history | 0 | 0 | 1 | 0 | 0 | 0 | 0 | 1 | 0 |
| 7 | Review evaluation | 1 | 1 | 0 | 0 | 0 | 0 | 1 | 0 | 0 |
| 8 | Seasonal-limited | 0 | 1 | 1 | 0 | 0 | 0 | 0 | 0 | 0 |
| | commodities | | | | | | | | | |
| 9 | Identical attribute | 0 | 0 | 0 | 1 | 0 | 0 | 0 | 1 | 0 |
| | purchased | | | | | | | | | |
| | commodities | | | | | | | | | |
| 10 | Purchase canceled | 0 | 0 | 1 | 0 | 0 | 1 | 1 | 0 | 0 |
| | commodities | | | | | | | | | |
| 11 | Strategic commodities | 0 | 1 | 0 | 0 | 0 | 1 | 0 | 0 | 0 |

| | | | | | | | | | |
|---|---|---|---|---|---|---|---|---|---|
| Total | 4 | 4 | 3 | 1 | 3 | 3 | 2 | 5 | 2 |

As shown in Table 2 above, in the second evaluation item group, the temporal food classification (No. 1) is “suitable for lunch”. Incidentally, the evaluation items from the new commodities (No. 2) to the strategic commodities (No. 11) are the same as the first evaluation item group. In addition, the health food F which was the object of determination in Table 1 is excluded.

As shown in Table 1 above, for food A, a total of 4 points are assigned to new commodities (No. 2), favorite commodities (No. 3), recommended commodities (No. 5), and review evaluation (No. 7) in order to satisfy the evaluation items. Food B is given a total of four points to satisfy the evaluation items for good-selling commodities (No. 4), review evaluation (No. 7), seasonal-limited commodities (No. 8) and strategic commodities (No. 11). Food C is given a total of three points to satisfy the evaluation items for search history (No. 6), seasonal-limited commodities (No. 8) and purchased canceled commodities (No. 10), while Food D is given one point to satisfy the evaluation items for identical attribute purchased commodities (No. 9).

For health foods E, a total of 3 points are assigned in order to satisfy the criteria for temporal food classification (No. 1), new commodities (No. 2), and good-selling commodities (No. 4). For health foods G, a total of 3 points are assigned in order to satisfy the criteria for recommended commodities (No. 5), purchase canceled commodities (No. 10), and strategic commodities (No. 11).

For supplement H, a total of 2 points are assigned to satisfy the criteria for review evaluation (No. 7) and purchase canceled commodities (No. 10). For supplement I, a total of 5 points are assigned to satisfy the criteria for favorite commodities (No. 3), good-selling commodities (No. 4), recommended commodities (No. 5), search history (No. 6), and identical attribute purchased commodities (No. 9). For supplement J, a total of 2 points are assigned to satisfy the criteria for good-selling commodities (No. 4) and recommended commodities (No. 5).

As described above, the supplement I having the highest total point of 5 points is regarded as the commodity having the highest priority and is excluded from the determination target in step S15.

Since eight types of commodities of foods (A to D), health foods (E, G) and supplements (H, J) remain as the determination targets (step S16), weighting is performed using the third group of evaluation items as the determination targets (step S17). In the same manner as in the first or second determination, the commodity having the highest priority is determined and excluded from the determination targets (step S18). Table 3 below shows a determination image when the third determination is performed using the third evaluation item group.

TABLE 3

| | | | | | | Health | Health | Supplement | Supplement |
|---|---|---|---|---|---|---|---|---|---|
| No. | Evaluation items | Food A | Food B | Food C | Food D | food E | food G | H | J |
| 1 | Temporal food | 0 | 1 | 0 | 1 | 0 | 0 | 0 | 0 |
| | classification (Snack) | | | | | | | | |
| 2 | New commodities | 1 | 0 | 0 | 0 | 1 | 0 | 0 | 0 |
| 3 | Favorite commodities | 1 | 0 | 0 | 0 | 0 | 0 | 0 | 0 |
| 4 | Good-selling | 0 | 1 | 0 | 0 | 1 | 0 | 0 | 1 |
| | commodities | | | | | | | | |
| 5 | Recommended | 1 | 0 | 0 | 0 | 0 | 1 | 0 | 1 |
| | commodities | | | | | | | | |
| 6 | Search history | 0 | 0 | 1 | 0 | 0 | 0 | 0 | 0 |
| 7 | Review evaluation | 1 | 1 | 0 | 0 | 0 | 0 | 1 | 0 |
| 8 | Seasonal-limited | 0 | 1 | 1 | 0 | 0 | 0 | 0 | 0 |
| | commodities | | | | | | | | |
| 9 | Identical attribute | 0 | 0 | 0 | 1 | 0 | 0 | 0 | 0 |
| | purchased | | | | | | | | |
| | commodities | | | | | | | | |
| 10 | Purchase canceled | 0 | 0 | 1 | 0 | 0 | 1 | 1 | 0 |
| | commodities | | | | | | | | |
| 11 | Strategic commodities | 0 | 1 | 0 | 0 | 0 | 1 | 0 | 0 |

| | | | | | | | | |
|---|---|---|---|---|---|---|---|---|
| Total | 4 | 5 | 3 | 2 | 2 | 3 | 2 | 2 |

As shown in Table 3 above, in the third evaluation item group, the temporal food classification (No. 1) is “suitable for snacks”. The evaluation items from the new commodities (No. 2) to the strategic commodities (No. 11) are the same as the first or second evaluation item group. In addition, supplement I which has been set as a determination target in Tables 1 and 2 is excluded.

As shown in Table 3 above, for food A, a total of 4 points are assigned to new commodities (No. 2), favorite commodities (No. 3), recommended commodities (No. 5), and review evaluation (No. 7) in order to satisfy the evaluation items. Food B is given a total of five points for time food classification (No. 1), good-selling commodities (No. 4), review evaluation (No. 7), seasonal-limited commodities (No. 8) and strategic commodities (No. 11) in order to satisfy the evaluation items, Food C is given a total of three points for search history (No. 6), seasonal-limited commodities (No. 8) and purchase canceled commodities (No. 10), and Food D is given a total of two points for temporal food classification (No. 1) and identical attribute purchased commodities (No. 9) in order to satisfy the evaluation items.

For health foods E, a total of 2 points are assigned to new commodities (No. 2) and good-selling commodities (No. 4) in order to satisfy the criteria. For health foods G, a total of 3 points are assigned to recommended commodities (No. 5), purchase canceled commodities (No. 10) and strategic commodities (No. 11) in order to satisfy the criteria.

For supplement H, a total of 2 points are assigned for review evaluation (No. 7) and purchase canceled commodities (No. 10) in order to satisfy the evaluation items, and for supplement J, a total of 2 points is assigned for good-selling commodities (No. 4) and recommended commodities (No. 5) in order to satisfy the evaluation items.

As described above, the food B having the highest total point of 5 points is regarded as the commodity having the highest priority, and is excluded from the determination target (step S18).

Here, since seven types of commodities of food (A, C, D), health food (E, G), and supplement (H, J) remain as the determination targets (step S19), weighting is performed using the fourth evaluation item group as the determination targets (step S20). Here, as in the first to third determinations, the commodities with the highest priority are determined and excluded from the determination targets (step S21). Table 4 below shows a determination image when the fourth determination is performed using the fourth evaluation item group.

TABLE 4

| | | | | | Health | Health | Supplement | Supplement |
|---|---|---|---|---|---|---|---|---|
| No. | Evaluation items | Food A | Food C | Food D | food E | food G | H | J |
| 1 | Temporal food | 0 | 1 | 1 | 0 | 0 | 0 | 0 |
| | classification (Dinner) | | | | | | | |
| 2 | New commodities | 1 | 0 | 0 | 1 | 0 | 0 | 0 |
| 3 | Favorite commodities | 1 | 0 | 0 | 0 | 0 | 0 | 0 |
| 4 | Good-selling | 0 | 0 | 0 | 1 | 0 | 0 | 1 |
| | commodities | | | | | | | |
| 5 | Recommended | 1 | 0 | 0 | 0 | 1 | 0 | 1 |
| | commodities | | | | | | | |
| 6 | Search history | 0 | 1 | 0 | 0 | 0 | 0 | 0 |
| 7 | Review evaluation | 1 | 0 | 0 | 0 | 0 | 1 | 0 |
| 8 | Seasonal-limited | 0 | 1 | 0 | 0 | 0 | 0 | 0 |
| | commodities | | | | | | | |
| 9 | Identical attribute | 0 | 0 | 1 | 0 | 0 | 0 | 0 |
| | purchased | | | | | | | |
| | commodities | | | | | | | |
| 10 | Purchase canceled | 0 | 1 | 0 | 0 | 1 | 1 | 0 |
| | commodities | | | | | | | |
| 11 | Strategic commodities | 0 | 0 | 0 | 0 | 1 | 0 | 0 |

| | | | | | | | |
|---|---|---|---|---|---|---|---|
| Total | 4 | 4 | 2 | 2 | 3 | 2 | 2 |

As shown in Table 4 above, in the fourth evaluation item group, the temporal food classification (No. 1) is “suitable for dinner”. Incidentally, the evaluation items from new commodities (No. 2) to strategic commodities (No. 11) are the same as those in the evaluation item groups of 1 to 3. Food B, which was the target of evaluation in Tables 1 to 3, is excluded.

As shown in Table 4 above, for food A, a total of 4 points are assigned to new commodities (No. 2), favorite commodities (No. 3), recommended commodities (No. 5), and review evaluation (No. 7) in order to satisfy the evaluation items. For Food C, a total of 4 points are assigned to satisfy the judgment items for temporal food classification (No. 1), search history (No. 6), seasonal-limited commodities (No. 8), and purchase canceled commodities (No. 10). For Food D, a total of 2 points are assigned to satisfy the evaluation items for temporal food classification (No. 1) and identical attribute purchased commodities (No. 9).

For health foods E, a total of 2 points are assigned to new commodities (No. 2) and good-selling commodities (No. 4) in order to satisfy the criteria. For health foods G, a total of 3 points are assigned to recommended commodities (No. 5), purchase canceled commodities (No. 10) and strategic commodities (No. 11) in order to satisfy the criteria.

For supplement H, a total of 2 points are assigned for review evaluation (No. 7) and purchase canceled commodities (No. 10) in order to satisfy the evaluation items, and for supplement J, a total of 2 points is assigned for good-selling commodities (No. 4) and recommended commodities (No. 5) in order to satisfy the evaluation items.

As described above, the commodities with the highest total points of 4 points are the two types of food A and food C. In this way, when there is more than one commodity with the highest point, the commodity that satisfies the evaluation item of “No.” higher than the above is selected. Here, since the food C satisfies the evaluation item of the temporal food classification (No. 1), whereas the food A does not satisfy the evaluation item of the temporal food classification (No. 1), the food C is regarded as the commodity having the highest priority and is excluded from the determination target (step S21).

Here, since six types of commodities of food (A, D), health food (E, G) and supplement (H, J) remain as the determination targets (step S22), weighting is performed again using the first evaluation item group as the determination targets (step S11). Here, as in the first to fourth determinations, the commodities with the highest priority are determined and excluded from the determination targets (step S12).

Hereinafter, with respect to the priority setting, steps S11 to S22 are repeated until all the commodities are excluded. As a result, the priorities were set in the order of health food F in the first place, supplement I in the third place, food C in the fifth place, food A in the fifth place, health food G in the sixth place, food D in the seventh place, health food E in the eighth place, supplement J in the ninth place, and supplement H in the tenth place.

(Commodity Determining Step)

As shown in FIG. 2, after the priority is set, the proposed commodity is determined based on the priority (step S07: Commodity determining step). The commodity determination is to determine the number of items of the proposed commodity and the order of proposals. Here, it is assumed that the number of items of the proposed commodity is five. The order of proposal of the proposed commodity is preferably the order of attributes of “main dish”, “main dish”, “side dish”, “seasoning”, and “juice”, but may be limited to some of the above attributes or may be added to other attributes. When the number of items is larger than the number of attributes, the selection is repeatedly performed according to the order of proposal. In order to explain this, in the present embodiment, the order of proposal of the proposed commodity is described as the order of four attributes of “main dish”, “main meal”, “sub-dish” and “others”.

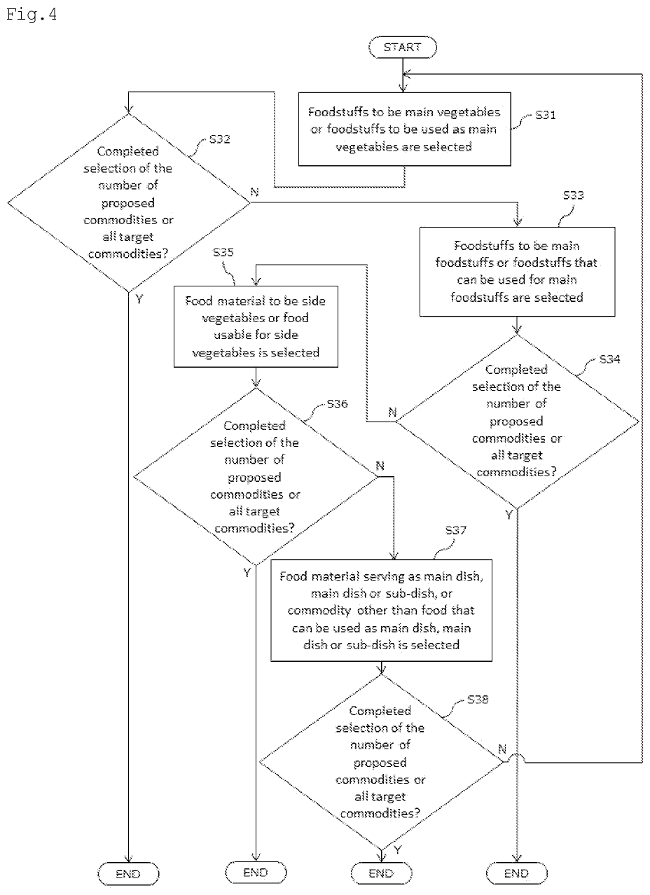

FIG. 4 shows a commodity decision flow diagram of a nutrition-related commodity recommendation method. As shown in FIG. 4, first, foodstuffs to be main vegetables or foodstuffs to be used as main vegetables are selected in step S31. Here, foodstuffs serving as main vegetables are meat and fish, and foodstuffs that can be used as main vegetables are foodstuffs and seasonings that are cooked together with meat and fish. In the present embodiment, the health food F, which is the food having the first priority and which can be used as a main vegetable, is selected.

When the number of items of the proposed commodity or the selection of all the target commodities is completed (step S32), the selection of the commodity is completed, but in the present embodiment, since the commodities of the five items are selected, the foodstuffs to be the main foodstuffs or the foodstuffs that can be used for the main foodstuffs are further selected (step S33). Here, foods serving as main foods are rice, bread, and the like, and foods that can be used as main foods are foods and seasonings that are cooked together with rice and bread.

In the present embodiment, the food B having the third priority and serving as the main food is selected. When the number of items of the proposed commodity or the selection of all the target commodities has been completed (step S34), the selection of the commodities is completed, but in the present embodiment, since the commodities of the five items are selected, the food material to be the side vegetables or the food usable for the side vegetables is further selected (step S35). Here, the foodstuffs serving as side vegetables are vegetables and the like, and the foodstuffs that can be used as side vegetables are foodstuffs and seasonings that are cooked together with the vegetables and the like. In the present embodiment, the health food G, which is the food having the priority of 6 and which can be used as a side dish, is selected.

When the selection of the number of items of the proposed commodity or all the target commodities is completed (step S36), the selection of the commodity is completed, but in the present embodiment, in order to select the commodities of the five items, a food material serving as a main dish, a main dish or a sub-dish, or a commodity other than a food that can be used as a main dish, a main dish or a sub-dish is further selected (step S37). Here, the above commodity is, for example, a supplement or a beverage. In this embodiment, the supplement I having the second priority is selected.

When the number of items of the proposed commodity or the selection of all the target commodities has been completed (step S38), the selection of the commodities is completed, but in the present embodiment, since the commodities of the five items are selected, the foodstuffs that become the main vegetables or the foodstuffs that can be used for the main vegetables are further selected (step S31). In the present embodiment, the food C having the fourth priority and serving as a main vegetable is selected.

In this embodiment, since the five items are selected, the selection of the number of items of the suggested item is completed (step S36). Specifically, it is decided to propose the health food F, the food B, the health food G, the supplement I, and the food C in this order.

(Commodity Adjustment Step)

After the proposed commodity is determined, the type of the proposed commodity can be manually adjusted (step S08: commodity adjustment step). In the commodity adjusting means step, the addition, change, and deletion of the item of the proposed commodity and the change of the order and design of the proposal can be performed by the hand of the service provider. It should be noted that the commodity adjustment is not necessarily performed, and, for example, in the present embodiment, the data output is performed without performing the adjustment.

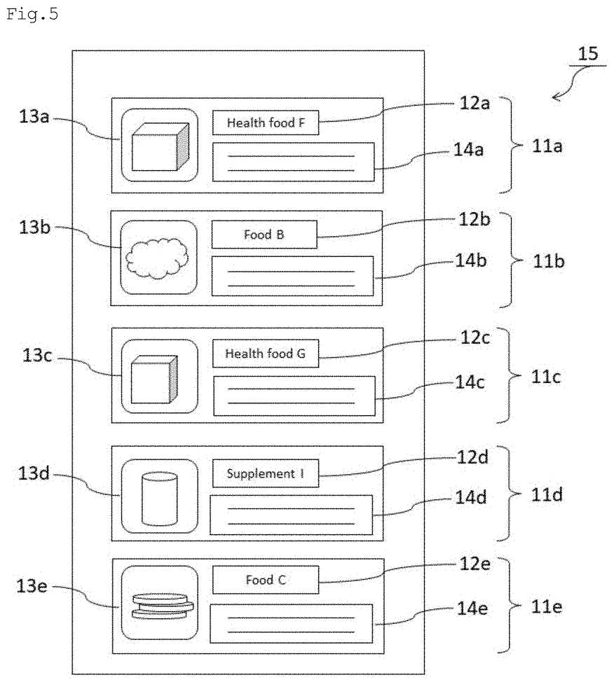

Finally, data relating to the proposed commodity is output (step S09). FIG. 5 is a screen display image diagram of the user terminal. As shown in FIG. 5, the commodity display columns (11a to 11e) are set on the display 15 of the terminal held by the user, and the health food F is displayed as the commodity name 12a in the commodity display column 11a at the top. In the commodity display field 11a, a commodity image 13a and a commodity description 14a are displayed to convey the image and attractiveness of the commodity.

Similarly, in the commodity display column 11b, the food item B is displayed as the commodity name 12b, and the commodity image 13b and the commodity description 14b are displayed. In the commodity display column 11c, the health food G is displayed as the commodity name 12c, and a commodity image 13c and a commodity description 14c are displayed. In the commodity display column 11d, the supplement I is displayed as the commodity name 12d, and the commodity image 13d and the commodity description 14d are displayed. In the commodity display column 11e, the food C is displayed as the commodity name 12e, and the commodity image 13e and the commodity description 14e are displayed.

In this manner, on the display 15, the commodity names (12a to 12e), the commodity images (13a to 13e), and the commodity descriptions (14a to 14e) are displayed in order of the health food F, the food B, the health food G, the supplement I, and the food C from the top, thereby realizing the commodity display that enhances the user's willingness to purchase the commodity.

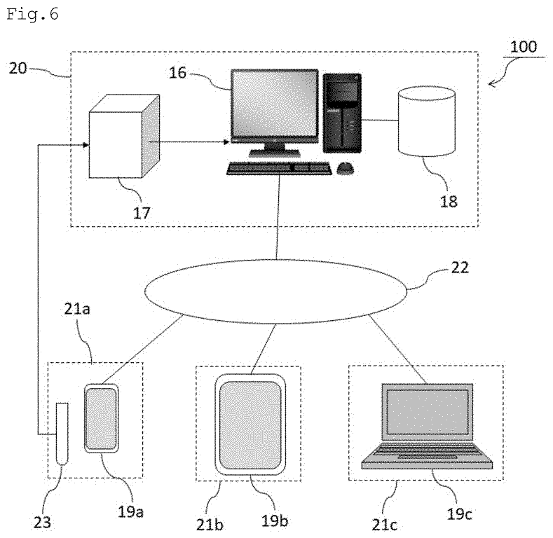

FIG. 6 is a schematic configuration diagram of the nutrition-related commodity recommendation system of Embodiment 1. As shown in FIG. 6, the nutrition-related commodity recommendation system 100 includes a management server 16, a measuring device 17, a smart phone 19a, a tablet terminal 19b, and a PC 19c, and the management server 16, the smart phone 19a, the tablet terminal 19b, and PC 19c can communicate with each other via the Internet 22. Further, the management server 16 includes a nutrition-related commodity recommendation device 1 (not shown) and a database 18.

The user 21a has the smartphone 19a, the user 21b has the tablet terminal 19b, and the user 21c has PC 19c. Thus, the user can utilize any terminal. Here, the user 21a will be described as an example.

The user 21a collects urine using the test kit 23 and sends the urine to the service provider 20. The service provider 20 measures the urine component of the user 21a in the measurement device 17, and inputs the measurement result to the management server 16. Data relating to the input measurement results is stored in the database 18.

The user 21a responds to the questionnaire provided by the management server 16 using the smartphone 19a, and transmits the questionnaire to the management server 16 using the Internet 22. The response results are stored in the database 18.

Based on the measurement result of the urine component and the answer result of the questionnaire, the management server 16 calculates a deficient nutrient in the body of the user using the nutrient information calculating means 4. The management server 16 narrows down and determines the proposed commodity by using the related commodity extraction means 5, the inappropriate commodity exclusion means 6, the priority setting means 7, and the commodity determining means 8 based on the calculated information on the nutrient. Further, the service provider 20 performs adjustment of the proposed commodity by using the commodity adjustment means 9 provided in the management server 16.

When the user 21a accesses a service (not shown) provided by the service provider 20 via the Internet 22, the management server 16 outputs data on the proposed commodity by the proposed commodity output means 10, and the proposed commodity is displayed on the display of the smartphone 19a held by the user 21a.

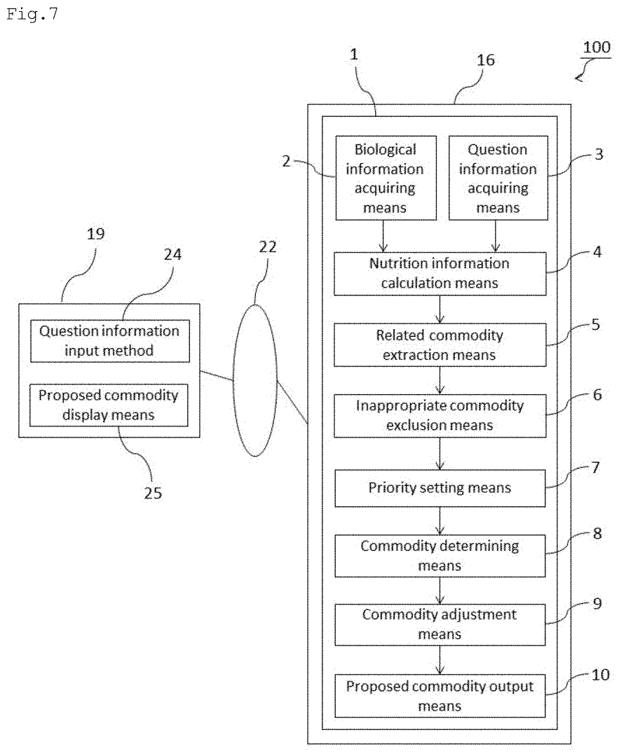

FIG. 7 is a functional block diagram of the nutrition-related commodity recommendation system according to Embodiment 1. As shown in FIG. 7, the nutrition-related commodity recommendation system 100 is a system in which a client terminal 19 on the user side and a management server 16 on the service provider side are connected via the internet 22 to transmit and receive data. The management server 16 includes a nutrition-related commodity recommendation device 1, and the nutrition-related commodity recommendation device 1 includes a biological information acquiring means 2, a question information acquiring means 3, a nutrition information calculation means 4, a related commodity extraction means 5, an inappropriate commodity exclusion means 6, a priority setting means 7, a commodity determining means 8, a commodity adjustment means 9, and a suggested commodity output means 10. The client terminal 19 further includes question information input means 24 for inputting question information of the user, and proposed commodity display means 25 for displaying proposed commodities on the display.

### Embodiment 2

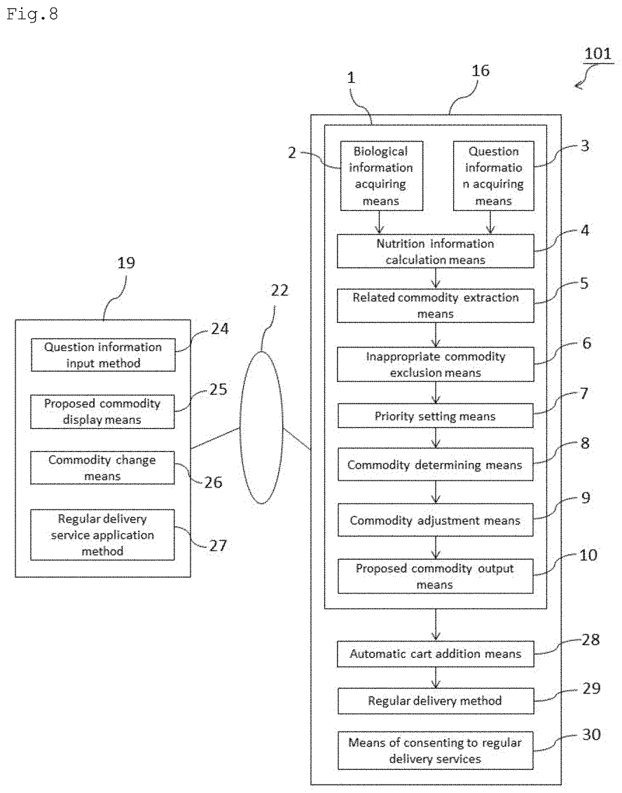

FIG. 8 shows a functional block diagram of the nutrition-related commodity recommendation system of Embodiment 2. As shown in FIG. 8, the nutrition-related commodity recommendation system 101 is a system in which a client terminal 19 on the user side and a management server 16 on the service provider side are connected via the Internet 22 to transmit and receive data. The management server 16 includes a nutrition-related commodity recommendation device 1, cart automatic addition means 28 for automatically adding suggested commodities to the user's cart in electronic commerce, periodic delivery means 29 for delivering commodities at predetermined dates or every elapse of a predetermined period, and regular delivery service acceptance means 30 for accepting an application for regular delivery service by the user, and the nutrition-related commodity recommendation device 1 includes biological information acquiring means 2, question information acquiring means 3, nutrition information calculation means 4, related commodity extraction means 5, inappropriate commodity exclusion means 6, priority setting means 7, commodity determining means 8, commodity adjustment means 9, and proposed commodity output means 10. In addition, the client terminal includes question information input means 24 for inputting question information of the user, proposed commodity display means 25 for displaying proposed commodities on the display, commodity change means 26 for excluding unnecessary commodities from proposed commodities automatically added to the cart or manually adding necessary commodities to the cart, and regular delivery service application means 27 for applying for periodic delivery of commodities.

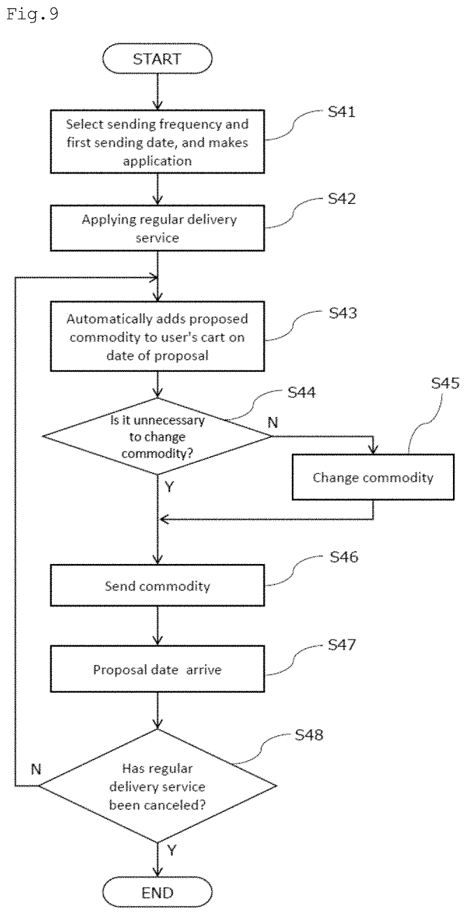

FIG. 9 shows a schematic flow diagram of the nutrition-related commodity recommendation system of Embodiment 2. As shown in FIG. 9, first, the user uses the regular delivery service application means 27 provided in the client terminal 19 to select the sending frequency and the first sending date, and makes an application in step S41. For example, items such as “once a week” or “once a month” are displayed on the display of the client terminal 19, and the user selects one of them. The selection of the first delivery date is a criterion for the date of commodity proposal and delivery in the regular delivery service.

In step S42, the regular delivery service accepting means 30 included in the management server 16 accepts the application when the application from the user is normally made, thereby applying the regular delivery service.

The management server 16 outputs the proposed commodity on the date of proposal by using the nutrition-related commodity recommendation device 1, and automatically adds the proposed commodity to the user's cart in the electronic commerce by using the cart automatic adding means 28 (step S43). Specifically, the proposed commodity is displayed on the display of the client terminal 19 in a state where the proposed commodity is automatically added to the cart by the proposed commodity display means 25 provided in the client terminal 19.

It is to be noted that acquisition of biological information by the biological information acquiring means 2 of the nutrition-related commodity recommendation device 1 is preferably performed by immersing the urine test paper in the urine of the user, and then capturing an image of the urine test paper using a camera means (not shown) provided in the client terminal 19 to measure the urine component.

In addition, in the same manner as in the nutrition-related commodity recommendation system 100 of Example 1, urine may be collected using an inspection kit and sent to a service provider, and a urine component of a user may be measured in a measurement device and a measurement result may be input into the management server 16. The question information acquiring means 3 of the nutrition-related commodity recommendation device 1 acquires the question information by transmitting the content input using the question information input means 24 of the client terminal 19 to the management server 16 via the Internet 22. The inputting of the question information is preferably performed before the proposal of the commodity on the commodity proposal date, and when the inputting of the question information is not completed on the commodity proposal date, the input of the question information is waited for and the proposal of the commodity is made. The inputting of the question information may be performed either before or after the application for the regular delivery service.

The user can confirm the proposed commodity and examine whether the commodity is necessary or unnecessary. When it is necessary to change the commodity such as exclusion of unnecessary commodities or addition of necessary commodities (step S44), the commodity is changed using the commodity changing means 26 (step S45). The change of the commodity needs to be made by a predetermined time limit, for example, “up to three days prior to the delivery of the commodity”.

The service provider sends the commodity to the user by using the periodic sending means 29 (step S46). Specifically, the periodic sending means 29 sends the commodity to the address of the user based on the information such as the name and address of the user acquired by the question information acquiring means 3. The user may set a destination other than the address.

Thereafter, when the proposal date further arrives (step S47), if the regular delivery service has not been cancelled (step S48), the commodity is proposed again (step S43). It is preferable that the proposal date is set to a date which is a predetermined period before the next scheduled sending date.

According to the nutrition-related commodity recommendation system 101 of Embodiment 2, it is possible to receive periodic commodity delivery only by once applying for the regular delivery service. Further, with respect to the commodity to be sent, the commodity matching the nutritional state or the like of the user is automatically proposed and automatically added to the cart of the user by the nutritional-related commodity recommendation device 1, so that the user can select the commodity matching the nutritional state or the preference of the user only by performing a very simple operation. As described above, according to the nutrition-related commodity recommendation system 101, the user can receive a high-quality regular delivery service with a simple operation.

### Other Embodiments

The related commodity extraction step (step S04), the inappropriate commodity exclusion step (step S05), the priority setting step (step S06), the commodity determining step (step S07), and the commodity adjustment step (step S08) are not limited to the order of Embodiment 1, and may be performed in any order.

### INDUSTRIAL APPLICABILITY

The present invention is useful as a technique for proposing a nutrition-related commodity based on biological information and the like.

### DESCRIPTION OF SYMBOLS

1 Nutrition-related commodity recommendation device
2 Biological information acquiring means
3 Question information acquiring means
4 Nutrition information calculation means
5 Related commodity extraction means
6 Inappropriate commodity exclusion means
7 Priority setting means
8 Commodity determining means
9 Commodity adjustment means
10 Proposed commodity output means
11a-11e Commodity display column
12a-12e Commodity name
13a-13e Commodity image
14a-14e Commodity description
15 Display
16 Management server
17 Measuring device
18 Database
19 Client terminal
19a Smartphone
19b Tablet terminal
19c PC (Personal computer)
20 Service providers
21a-21c users
22 Internet
23 Inspection kit
24 Question information input method
25 Proposed commodity display means
26 Commodity change means
27 Regular delivery service application method
28 Automatic cart addition means
29 Regular delivery method
30 Means of consenting to regular delivery services
100,101 Nutrition-related commodity recommendation system

1. A nutrition-related commodity recommendation device for proposing commodities related to nutritional conditions of users comprising:
a biological information acquiring means for acquiring biological information of the users,
a question information acquiring means for acquiring the user's question information,
a nutrition information calculation means for calculating nutrition information on excess and deficiency of nutrients in the user's body based on the biological information and the question information,
a related commodity extraction means for extracting commodity groups related to excess and deficiency nutrients based on the calculated nutritional information,
an inappropriate commodity exclusion means for excluding inappropriate commodities that are not suitable for the users from the commodity groups,
a priority setting means for setting a priority for each commodity of the commodity groups from which the inappropriate commodities have been excluded,
a commodity determining means for determining proposed commodities on a basis of the priority, and
a proposed commodity output means for outputting data relating to the proposed commodities.

2. The nutrition-related commodity recommendation device according to claim 1, wherein the inappropriate commodity exclusion means determines and excludes the inappropriate commodities which are not suitable for the users from the commodity group based on the past proposal history and the commodities purchase history relating to the users.

3. The nutrition-related commodity recommendation device according to claim 2, wherein the inappropriate commodity exclusion means excludes the commodity based on at least one of allergy content of the user, age of the user, and gender of the user.

4. The nutrition-related commodity recommendation device according to claim 1, wherein the priority setting means sets priorities for each commodity using at least one of the following evaluation items: time food classification, new commodities, good-selling commodities, recommended commodities, strategic commodities, seasonal-limited commodities, and sale commodities, and weightings thereof.

5. The nutrition-related commodity recommendation device according to claim 4, wherein the priority setting means sets priorities for each commodity using further at least one of the following evaluation items: a commodity to be searched by the user, a favorite commodity to be registered by the user, a commodity having the identical attribute as a commodity purchased by the user, and a commodity to be canceled for purchase by the user, and weightings thereof.

6. The nutrition-related commodity recommendation device according to claim 1, wherein the commodity determining means determines the number of items of the proposed commodities and the order of proposals.

7. The nutrition-related commodity recommendation device according to claim 1, wherein the biological information is body fluid component information of the user obtained by an examination including urinalysis, and the nutritional information is information relating to nutrients including vitamins and minerals.

8. A nutrition-related commodity recommendation method for proposing commodities related to nutritional conditions of users comprising:
a biological information acquiring step for acquiring biological information of the users,
a question information acquiring step for acquiring the user's question information,
a nutrition information calculation step for calculating nutrition information on excess and deficiency of nutrients in the user's body based on the biological information and the question information,
a related commodity extraction step for extracting commodity groups related to excess or deficiency nutrients based on the calculated nutritional information,
an inappropriate commodity exclusion step for excluding inappropriate commodities that are not suitable for the users from the commodity groups,
a priority setting step for setting a priority for each commodity of the commodity groups from which the inappropriate commodities have been excluded,
a commodity determining step for determining proposed commodities on a basis of the priority, and
a proposed commodity output step for outputting data relating to the proposed commodities.

9. A nutrition-related commodity recommendation program, wherein the program makes a computer perform the nutritional information calculation step, the related commodity extraction step, the inappropriate commodity exclusion step, the priority setting step, the commodity determining step in claim 8.

10. A nutrition-related commodity recommendation system, in which a client terminal on a user side and a server on a service provider side are connected via a network to transmit and receive data, comprising:
the server comprises the nutrition-related commodity recommendation device according to claim 1,
the client terminal comprises a means for inputting question information of users and a means for displaying proposed commodities on a display.

11. The nutrition-related commodity recommendation system according to claim 10, wherein the server further comprises a means for automatically adding the proposed commodity to a user's cart in an electronic commerce transaction, and
wherein the client terminal further comprises commodity changing means for removing unnecessary commodities from the proposed commodities automatically added to the cart or manually adding necessary commodities to the cart.

12. The nutrition-related commodity recommendation system according to claim 11, wherein the client terminal further comprises a periodic service application means for applying periodic delivery service, and
wherein the server further comprising an acceptance means for accepting an application for the periodic service by the user and a means for sending commodities on a specified date or every time a specified period has passed.
